(19) United States
(12) Patent Application Publication
(10) Pub. No.: US 2009/0275854 A1
(43) Pub. Date: Nov. 5, 2009

(54) SYSTEM AND METHOD OF MONITORING PHYSIOLOGIC PARAMETERS BASED ON COMPLEX IMPEDANCE WAVEFORM MORPHOLOGY

(76) Inventors: Todd M. Zielinski, Minneapolis, MN (US); Douglas A. Hettrick, Andover, MN (US); Eduardo N. Warman, Maple Grove, MN (US)

Correspondence Address: MEDTRONIC, INC., 710 MEDTRONIC PARKWAY NE, MINNEAPOLIS, MN, 55432-9924, US

(21) Appl. No.: 12/112,655

(22) Filed: Apr. 30, 2008

(51) Int. Cl. A61B 5/053

(52) U.S. Cl. 600/547

(57) ABSTRACT

Changes in physiologic parameters may be detected in a patient by measuring the impedance of a tissue segment located in a selected electrode vector field, storing baseline impedance information based on the measured impedance, detecting changes in impedance characteristics from the baseline impedance information, and providing alerts for changes in the physiologic parameters based on the detected changes in impedance characteristics. In some situations, detecting the changes in impedance characteristics involves detecting changes in morphology of an impedance waveform, such as a cardiac component of an impedance waveform, a respiratory component of an impedance waveform, and the phase angle of the complex impedance.

### CROSS-REFERENCE TO RELATED APPLICATION(S)

Reference is hereby made to U.S. application Ser. No. ______ filed on even date herewith, for “Multi-Frequency Impedance Monitoring System” by T. Zielinski, D. Hettrick and S. Sarkar (Attorney Docket No. P0024382.00), which is hereby incorporated by reference in its entirety.

### BACKGROUND

The present invention relates to systems and methods for measuring intrathoracic impedance (intracardiac, intravascular, subcutaneous, etc.) in an implantable medical device (IMD) system, and for providing clinical analysis based on the impedance morphology of cardiac and/or respiratory waveforms.

In systems employing IMDs such as pacemakers, defibrillators, and others, it has proven beneficial to provide the ability to measure intrathoracic impedance. Intrathoracic impedance measuring is performed by monitoring the voltage differential between pairs of spaced electrodes as current pulses are injected into those same leads or into other electrodes. Changes in the measured intrathoracic impedance may indicate certain disease conditions that can be addressed by delivery of therapy or alarm notification, for example. The efficacy of impedance monitoring to evaluate and monitor pulmonary edema and worsening congestive heart failure has been demonstrated in the OptiVol® Fluid Status Monitoring system provided by Medtronic, Inc. of Minneapolis, Minn.

Further improvements in the ability of an intrathoracic impedance measuring system monitor physiologic parameters to assist in identifying disease conditions would be useful.

### SUMMARY

Changes in physiologic parameters may be detected in a patient by measuring the impedance of a tissue segment located in a selected electrode vector field, storing baseline impedance information based on the measured impedance, detecting changes in impedance characteristics from the baseline impedance information, and providing alerts for changes in the physiologic parameters based on the detected changes in impedance characteristics. In some situations, detecting the changes in impedance characteristics involves detecting changes in morphology of an impedance waveform, such as a cardiac component of an impedance waveform, a respiratory component of an impedance waveform, and the phase angle of the complex impedance.

### BRIEF DESCRIPTION OF THE DRAWINGS

FIG. 1 is a diagram illustrating an impedance waveform measured between two electrodes positioned cutaneously, subcutaneously, intravascularly, intracardially, or any combination of these.

FIG. 2 is a diagram illustrating a three element electrical equivalent model of tissue impedance.

FIG. 3A is a phasor diagram, and FIG. 3B is a schematic illustration depicting the real and reactive components of a complex impedance waveform in a normal (non-diseased) tissue segment.

FIG. 4A is a phasor diagram, and FIG. 4B is a schematic illustration depicting the real and reactive components of a complex impedance waveform in a diseased tissue segment.

FIG. 5 is a graph illustrating the results of an acute animal model during drug interventions designed to change the morphology of the cardiac component of an impedance waveform.

FIGS. 6A and 6B are graphs illustrating waveform morphologies from an acute animal model of left ventricular ischemia induced by full occlusion of the left anterior descending (LAD) coronary artery.

FIG. 7 is a graph illustrating impedance waveform morphologies during one cardiac cycle in a porcine model of pulmonary edema.

FIG. 8 is a diagram illustrating an exemplary electrode vector configuration for monitoring physiologic parameters associated with cardiac and respiratory function.

FIGS. 9A-9B together are a flow diagram illustrating a method of detecting changes in the morphology of a cardiac impedance waveform and providing alerts for clinical conditions based on the detected morphology changes.

FIGS. 10A-10B together are a flow diagram illustrating a method of detecting changes in the morphology of a respiratory impedance waveform and providing alerts for clinical conditions based on the detected morphology changes.

FIGS. 11A-11B together are a flow diagram illustrating a method of detecting changes in the morphology of the phase component of a cardiac impedance waveform and providing alerts for clinical conditions based on the detected morphology changes.

### DETAILED DESCRIPTION

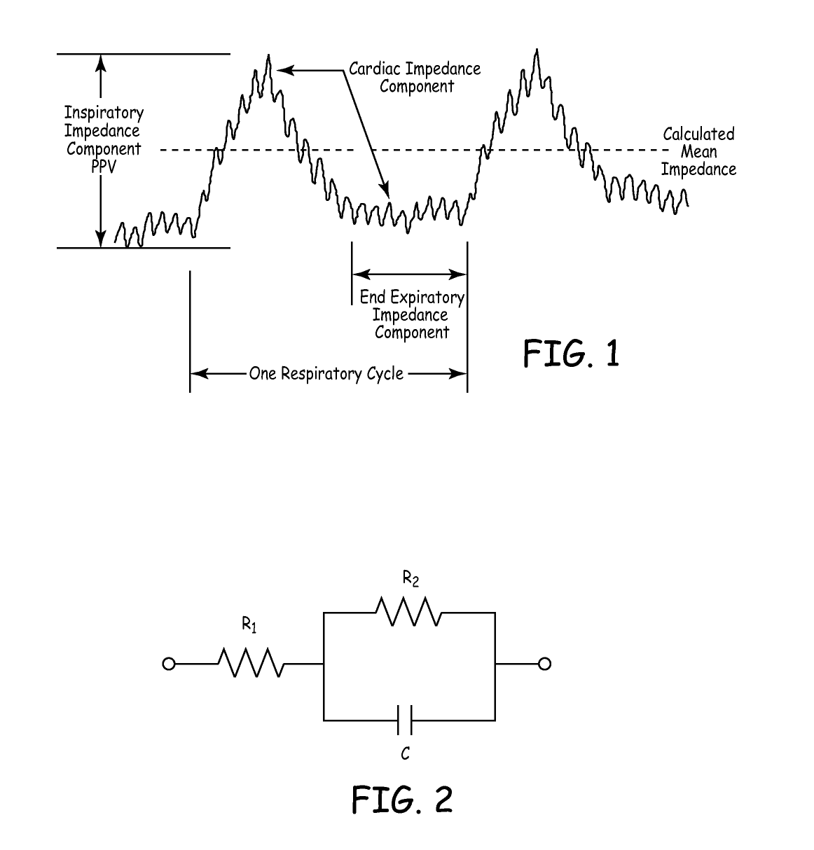

FIG. 1 is a diagram illustrating an impedance waveform measured between two electrodes positioned cutaneously, subcutaneously, intravascularly, intracardially, or any combination of these. The impedance waveform includes a high frequency cardiac component superimposed on a low frequency respiratory component and a calculated DC or mean component, and is shown over a period of two positive pressure ventilation (PPV) respiratory cycles. The morphologies of both the cardiac and respiratory components of the impedance waveform include real (R) resistive and imaginary (−Xc) reactive components. The real component of the impedance waveform is independent of the applied stimulation current frequency, and represents the resistive properties of tissue that consist primarily of extracellular space and fluid such as blood or plasma. The imaginary reactive component of the impedance waveform is dependent on the stimulation current frequency, and represents the capacitive properties of tissue that consist primarily of the cellular membranes of muscle tissue.

When more blood volume enters an electrode vector field, the impedance is primarily resistive, and due to the high conductivity of blood, the magnitude of impedance decreases. When more muscle tissue enters the electrode vector field such as during end systole of the cardiac cycle, the impedance is primarily reactive due to the capacitive properties of muscle tissue. Using both the impedance magnitude and the related phase angle between the real and imaginary reactive components, an indicator of cardiac function during a specific period of time during the cardiac cycle can be obtained and analyzed with clinical utility. This analysis may also be applicable to other organs, such as to monitor the onset or progression of disease such as during organ transplant or the like.

FIG. 2 is a diagram illustrating a three element electrical equivalent model of tissue impedance. R1 represents the resistive component of extracellular fluid and blood. R2 and C represent a cellular membrane of a specific tissue segment. At low injection current frequencies, the equivalent circuit is primarily resistive, so that the resistance of the tissue (RT) is equal to R1+R2. As the injection current frequency increases, the phase angle (φ) of the measured impedance increases and the reactive element C provides additional information regarding the characteristics of the selected tissue segment. During the onset or progression of disease, such as ischemia, worsening heart failure, etc., the attributes of each component change, and are therefore reflected in the waveform of the measured complex impedance signal.

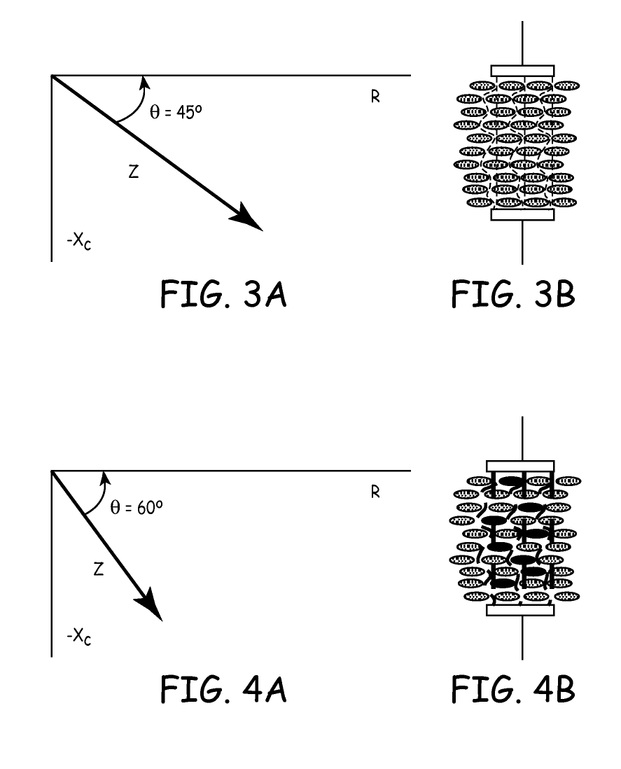

FIG. 3A is a phasor diagram, and FIG. 3B is a schematic illustration depicting the real and reactive components of a complex impedance waveform in a normal (non-diseased) tissue segment. FIG. 3B illustrates current flowing the resistive (extracellular fluid and blood) and reactive (cellular membrane) components of the tissue segment in a relatively uniform manner, so that the phase angle of the complex impedance waveform shown in FIG. 3A is about 45 degrees.

FIG. 4A is a phasor diagram, and FIG. 4B is a schematic illustration depicting the real and reactive components of a complex impedance waveform in a diseased tissue segment. As tissue becomes diseased, the current distribution through the tissue segment changes as a function of the change in the reactive (capacitive) and resistive properties of the tissue segment. In the example shown in FIGS. 4A and 4B, the tissue has become more reactive due to degradation of the cellular wall, so that the phase angle of the complex impedance waveform shown in FIG. 4A has increased to about 60 degrees. The relationship of the complex impedance waveform and its components as a function of resistance, capacitance and injection current frequency are given by the following equations:

$$\begin{matrix}Z = \sqrt{( {R}^{2} + {X}_{c}^{2} )} & ( Eq . 1 ) \\ {X}_{c} = \frac{1}{2 π fC} & ( Eq . 2 ) \\ θ = {tan}^{- 1} ( \frac{{X}^{c}}{R} ) & ( Eq . 3 )\end{matrix}$$

In the following discussion, it should be understood that references to impedance may refer to the real portion of impedance, the reactive portion of impedance, or the phase of the complex impedance, as appropriate.

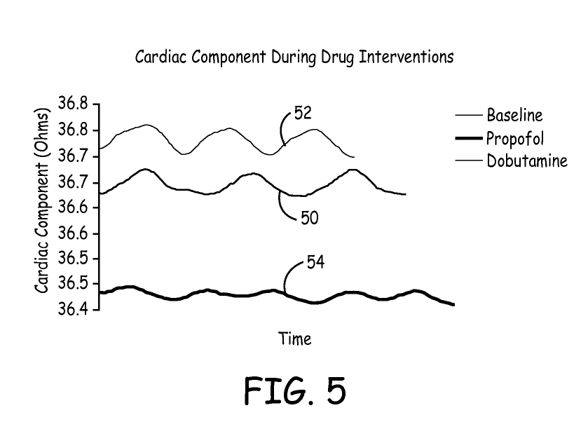

FIG. 5 is a graph illustrating the results of an acute animal model during drug interventions designed to change the morphology of the cardiac component of an impedance waveform. The impedance waveforms shown in FIG. 5 were obtained from a subcutaneous electrode vector configuration. Curve 50 shows a baseline impedance waveform morphology, with no drug intervention. Curve 52 shows an impedance waveform morphology with a dobutamine drug intervention, which created a condition of increased cardiac contractility. This is represented in curve 52 by an increase in both the magnitude and the maximal time derivative or slope of the impedance waveform morphology in comparison to baseline curve 50. Curve 54 shows an impedance waveform morphology with a propofol drug intervention, which created a condition of decreased cardiac contractility and decreased left ventricular afterload. This is represented in curve 54 by a decrease in both the magnitude and the slope of the impedance waveform morphology in comparison to baseline curve 50. Moreover, the increase in the mean impedance during the dobutamine drug intervention (curve 52) indicates that more blood volume is leaving the electrode vector field, while the decrease in the mean impedance during the propofol drug intervention (curve 54) indicates that more blood volume has entered the electrode vector field.

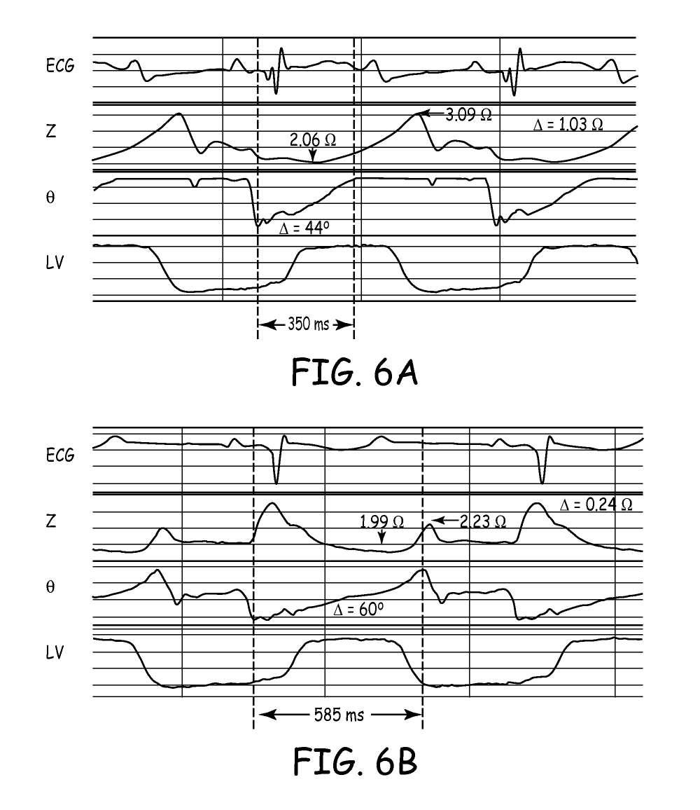

FIGS. 6A and 6B are graphs illustrating waveform morphologies from an acute animal model of left ventricular ischemia induced by full occlusion of the left anterior descending (LAD) coronary artery. Specifically, FIG. 6A shows waveform morphologies during normal coronary artery perfusion, and FIG. 6B shows waveform morphologies after five minutes of LAD occlusion. As shown, the waveform morphologies of the impedance (Z) and phase angle (θ) change during the acute stage of decreased coronary perfusion. During this stage, cellular ischemia theoretically leads to changes in the tissue characteristics, leading to waveform morphology changes in the impedance and phase angle waveforms, including changes in magnitude, mean, slope and timing intervals between minimum and maximum points. For example, in the example shown in FIGS. 6A and 6B, several changes in these characteristics can be observed as changing, including (a) the maximum peak impedance magnitude (changing from 3.09 Ohms to 2.23 Ohms), (b) the difference between the minimum peak impedance magnitude and the maximum peak impedance magnitude (changing from 1.03 Ohms to 0.24 Ohms), (c) the difference between the minimum peak phase angle and the maximum peak phase angle (changing from 44 degrees to 60 degrees), and (d) the time between a minimum peak phase angle and a maximum peak phase angle (changing from 350 milliseconds to 585 milliseconds).

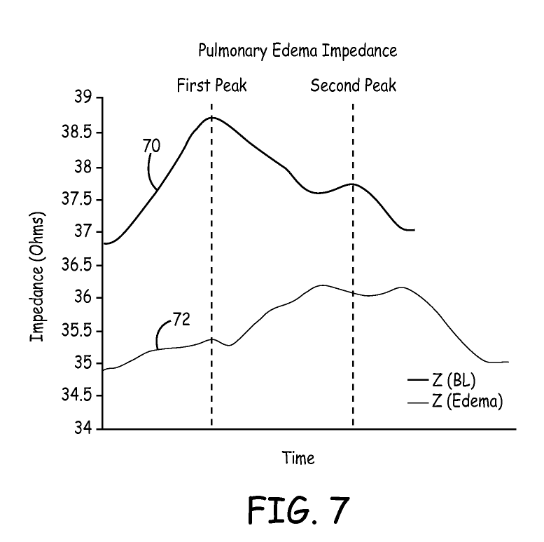

FIG. 7 is a graph illustrating impedance waveform morphologies during one cardiac cycle in an animal model of acute pulmonary edema. The impedance waveforms were filtered with a 2 Hertz low pass filter. The impedance waveforms were obtained with a bipolar electrode vector configuration including electrodes placed within the right ventricle and subcutaneously near the left clavicle, respectively. This electrode configuration is similar to that used in commercial products that monitor a fluid index based on intrathoracic impedance, such as the OptiVol® Fluid Status Monitoring system provided by Medtronic, Inc. of Minneapolis, Minn. Curve 70 shows a baseline impedance waveform morphology, and curve 72 shows an impedance waveform morphology during a condition of pulmonary edema. As shown, the impedance waveform morphology during the condition of pulmonary edema (curve 72) changed significantly in its peak amplitude, slope, mean impedance and cardiac cycle duration in comparison to the baseline condition (curve 70). The impedance waveform morphology changed between the first and second peaks, and during this time pulmonary edema was confirmed. Similar impedance waveform morphology changes may also be observed in a patient with dilated cardiomyopathy.

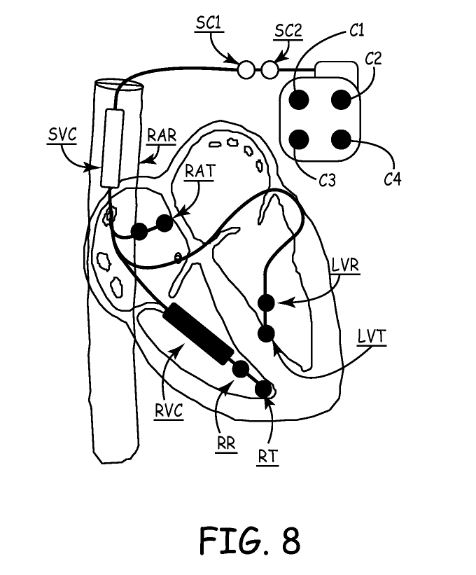

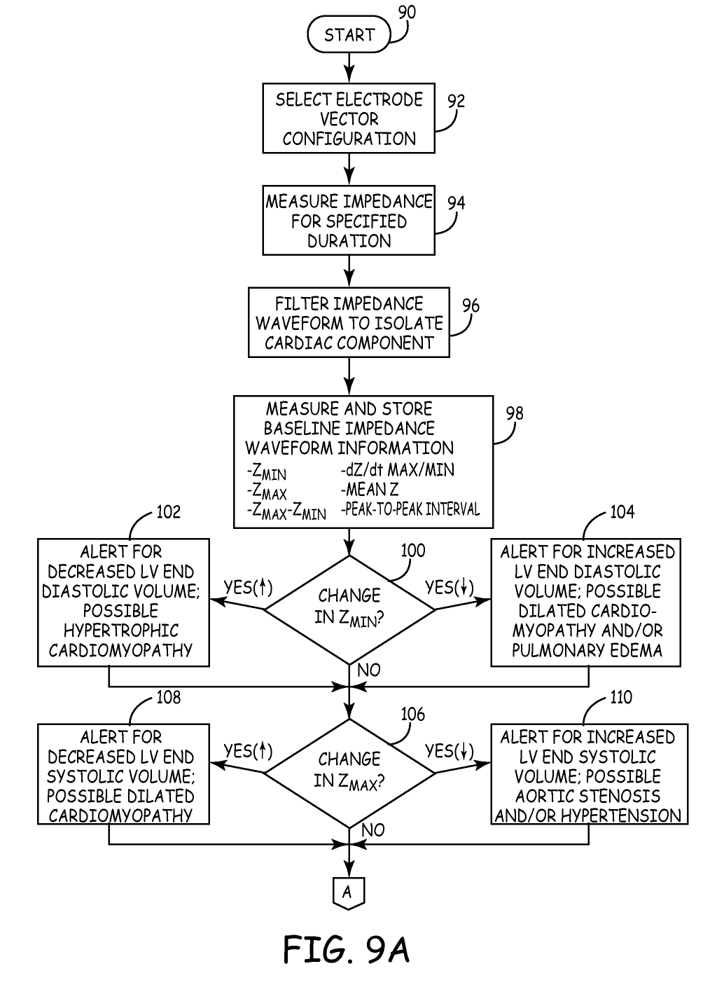

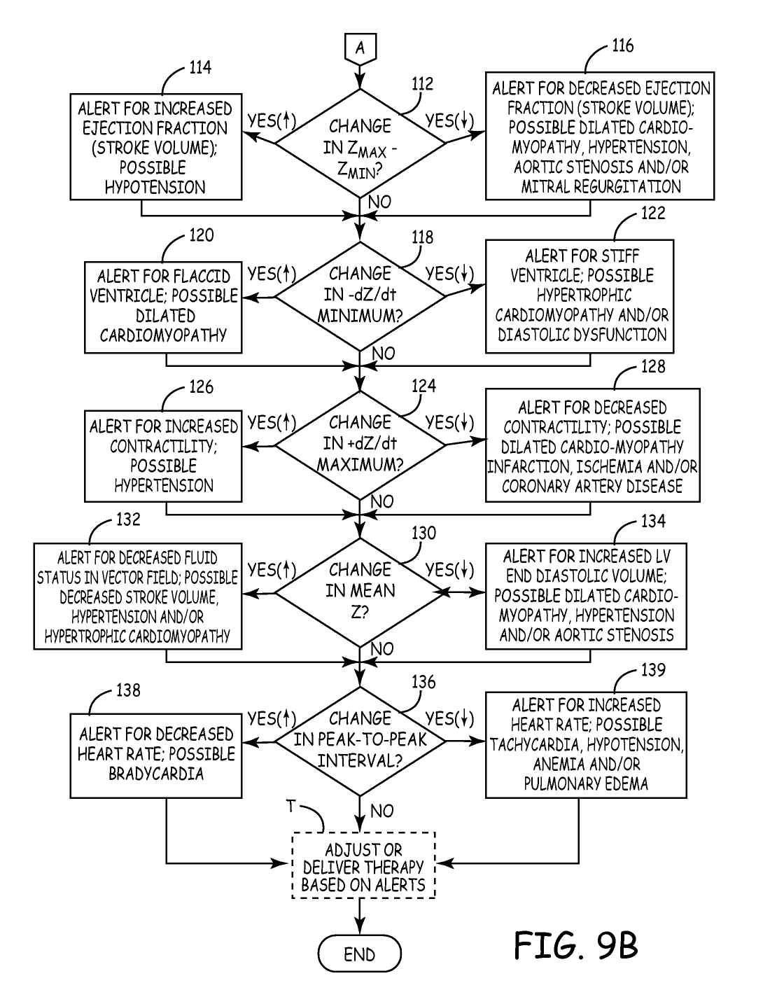

The combination of different waveform morphology changes and different selected electrode vectors can provide information about a variety of clinical conditions. FIG. 8 is a diagram illustrating exemplary intracardiac, intravascular and subcutaneous electrode locations that can be used for various bipolar, tripolar or quadripolar electrode configurations. Electrodes shown in FIG. 8 are superior vena cava coil SVC, right ventricular coil RVC, right ventricular ring RR, right ventricular tip RT, right atrial ring RAR, right atrial tip RAT, left ventricular ring LVR and left ventricular tip LVT (both located in the great cardiac vein or another suitable cardiac vein), subclavian vein electrodes SC1 and SC2 (which may be located in other suitable locations), and subcutaneous electrodes C1, C2, C3 and C4 on the device housing or can. An impedance can be measured in a tissue segment located in an electrode vector field between electrodes by injecting a current between selected electrodes, measuring a voltage between selected electrodes, and determining the impedance based on the injected current and the measured voltage. The impedance may change due to a change in the characteristics of the tissue in the electrode vector field (such as degradation of the cellular wall due to disease) or due to a change in the distance between electrodes (such as may be observed between the left ventricle and the right ventricle, which is representative of stroke volume). FIGS. 9A-9B, 10A-10B and 11A-11B are flow diagrams illustrating methods of detecting impedance waveform morphology changes for electrode vector configurations of FIG. 8, and for providing alerts for various changes in physiologic parameters based on the detected morphology changes. These methods are described in detail below.

FIGS. 9A-9B together are a flow diagram illustrating a method of detecting changes in the morphology of a cardiac impedance waveform and providing alerts for changes in physiologic parameters based on the detected morphology changes. Upon starting the method (box 90), an electrode vector configuration is selected (box 92) to measure impedance in a tissue segment located in the vector field. For the method steps of FIGS. 9A-9B, an electrode vector configuration selected from the electrodes located as shown in FIG. 8 is selected. Impedance is then measured for a specified duration (box 94), and the impedance waveform is filtered to isolate the cardiac component of the impedance (box 96). This filtering step is achieved by filtering out the low frequency respiratory component of impedance with a high pass filter of some kind. The impedance waveform is then analyzed to measure and store baseline impedance waveform information (box 98). This information may include (but is not limited to) a minimum peak impedance magnitude (Z_{MIN}), a maximum peak impedance magnitude (Z_{MAX}), a minimum to maximum impedance magnitude (Z_{MAX}-Z_{MIN}), a minimum negative slope (−dZ/dt) of the impedance waveform, a maximum positive slope (+dZ/dt) of the impedance waveform, a mean impedance, and a peak-to-peak time interval (which may involve a time interval between positive peaks (peaks), between negative peaks (nadirs), between a peak and a nadir, between a peak or a nadir and a characteristic of another monitored signal such as an ECG, between a point of maximum or minimum slope of the impedance waveform and a peak or a nadir, or others). Once this baseline impedance waveform information is determined and stored, changes in the impedance waveform with respect to the baseline values of these parameters may be detected and analyzed to provide alerts for various changes in physiologic parameters, as explained by the examples given below.

A change in the minimum peak impedance magnitude may be detected as indicated by step 100, to monitor left ventricle end diastolic volume at end expiration. If the minimum peak impedance magnitude has increased by an amount greater than a threshold, this indicates decreased left ventricle end diastolic volume. In this case, an alert may accordingly be provided to indicate the decreased left ventricle end diastolic volume (box 102). This alert may provide an indication to a clinician of possible hypertrophic cardiomyopathy or other manifestations of left ventricle dilation associated with new or worsening heart failure, for example. If the minimum peak impedance magnitude has decreased by an amount greater than a threshold, this indicates increased left ventricle end diastolic volume. In this case, an alert may accordingly be provided to indicate the increased left ventricle end diastolic volume (box 104). This alert may provide an indication to a clinician of possible dilated cardiomyopathy and/or pulmonary edema, for example.

A change in the maximum peak impedance magnitude may be detected as indicated by step 106, to monitor left ventricle end systolic volume at end expiration. If the maximum peak impedance magnitude has increased by an amount greater than a threshold, this indicates decreased left ventricle end systolic volume. In this case, an alert may accordingly be provided to indicate the decreased left ventricle end systolic volume (box 108). This alert may provide an indication to a clinician of possible dilated cardiomyopathy, for example. If the maximum peak impedance magnitude has decreased by an amount greater than a threshold, this indicates increased left ventricle end systolic volume. In this case, an alert may accordingly be provided to indicate the increased left ventricle end systolic volume (box 110). This alert may provide an indication to a clinician of possible aortic stenosis and/or hypertension, for example.

A change in the minimum to maximum impedance magnitude may be detected as indicated by step 112, to monitor stroke volume. If the minimum to maximum impedance magnitude has increased by an amount greater than a threshold, this indicates that the left ventricle has an increased ejection fraction. In this case, an alert may accordingly be provided to indicate the increased ejection fraction (box 114). This alert may provide an indication to a clinician of possible hypotension, for example. If the minimum to maximum impedance magnitude has decreased by an amount greater than a threshold, this indicates that the left ventricle has a decreased ejection fraction. In this case, an alert may accordingly be provided to indicate the decreased ejection fraction (box 116). This alert may provide an indication to a clinician of possible dilated cardiomyopathy, hypertension, aortic stenosis and/or mitral regurgitation, for example.

A change in the minimum negative slope of the impedance waveform may be detected as indicated by step 118, to monitor left ventricle lusitropic function or relaxation. If the minimum negative slope of the impedance waveform has increased by an amount greater than a threshold, this indicates a flaccid ventricle. In this case, an alert may accordingly be provided to indicate the flaccid ventricle (box 120). This alert may provide an indication to a clinician of possible dilated cardiomyopathy, for example. If the minimum negative slope of the impedance waveform has decreased by an amount greater than a threshold, this indicates a stiff ventricle. In this case, an alert may accordingly be provided to indicate the stiff ventricle (box 122). This alert may provide an indication to a clinician of possible hypertrophic cardiomyopathy and/or diastolic dysfunction, for example.

A change in the maximum positive slope of the impedance waveform may be detected as indicated by step 124, to monitor left ventricle inotropic contractility. If the maximum positive slope of the impedance waveform has increased by an amount greater than a threshold, this indicates increased contractility. In this case, an alert may accordingly be provided to indicate the increased contractility (box 126). This alert may provide an indication to a clinician of possible hypotension, for example. If the maximum positive slope of the impedance waveform has decreased by an amount greater than a threshold, this indicates decreased contractility. In this case, an alert may accordingly be provided to indicate the decreased contractility (box 128). This alert may provide an indication to a clinician of possible dilated cardiomyopathy, acute myocardial infarction, ischemia and/or coronary artery disease, for example.

A change in the mean impedance may be detected as indicated by step 130, to monitor fluid status in the vector field. If the mean impedance has increased by an amount greater than a threshold, this indicates decreased stroke volume due to decreased fluid in the vector field. In this case, an alert may accordingly be provided to indicate the decreased stroke volume (box 132). This alert may provide an indication to a clinician of possible hypertension and/or hypertrophic cardiomyopathy, for example. If the mean impedance has decreased by an amount greater than a threshold, this indicates increased left ventricle end diastolic volume. In this case, an alert may accordingly be provided to indicate the increased left ventricle end diastolic volume (box 134). This alert may provide an indication to a clinician of possible dilated cardiomyopathy, hypertension and/or aortic stenosis, for example.

A change in the peak-to-peak interval (involving a time interval between positive peaks in the example given) may be detected as indicated by step 136, to monitor heart rate. If the peak-to-peak interval has increased by an amount greater than a threshold, this indicates decreased heart rate. In this case, an alert may accordingly be provided to indicate the decreased heart rate (box 138). This alert may provide an indication to a clinician of possible bradycardia, for example. If the peak-to-peak interval has decreased by an amount greater than a threshold, this indicates increased heart rate. In this case, an alert may accordingly be provided to indicate the increased heart rate (box 139). This alert may provide an indication to a clinician of possible tachycardia, hypotension, anemia and/or pulmonary edema, for example.

Although the description above indicates that a clinician reviews an alert indicating a change in a physiologic parameter to determine whether a clinical condition exists and a therapy may be needed, the system and method of the present invention may be employed to automatically trigger an alert for a clinical condition (or a number of possible clinical conditions) and to adjust or deliver an appropriate therapy, as desired for a particular patient environment. Box T illustrates the optional adjustment or delivery of therapy in response to generated alerts.

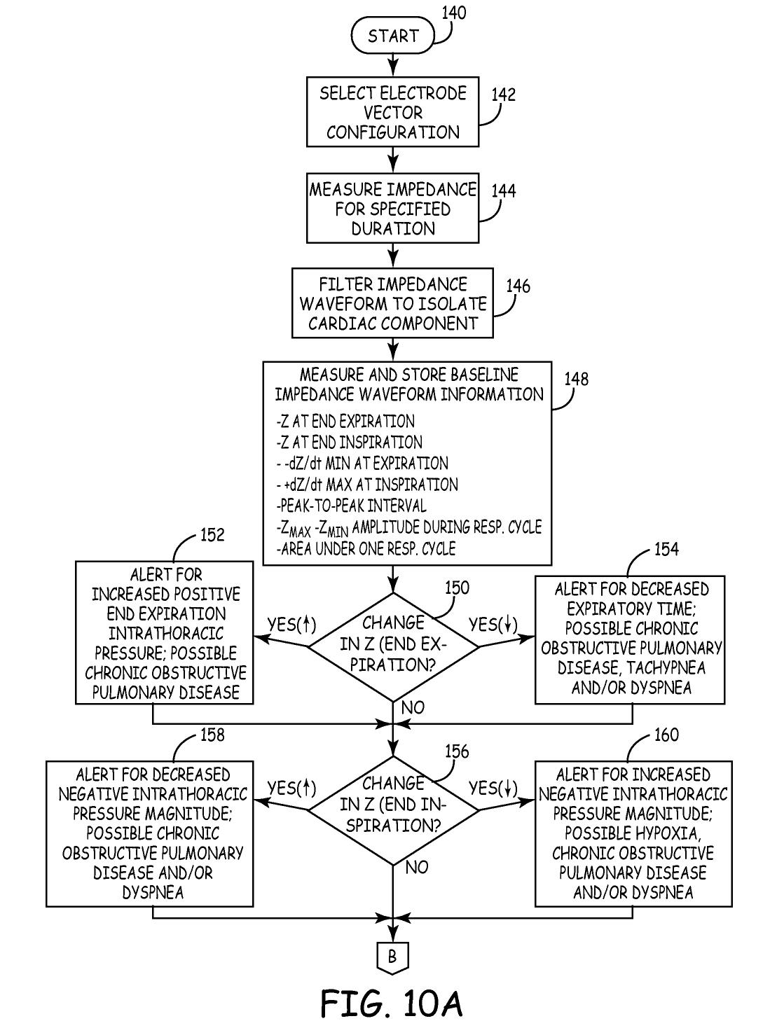

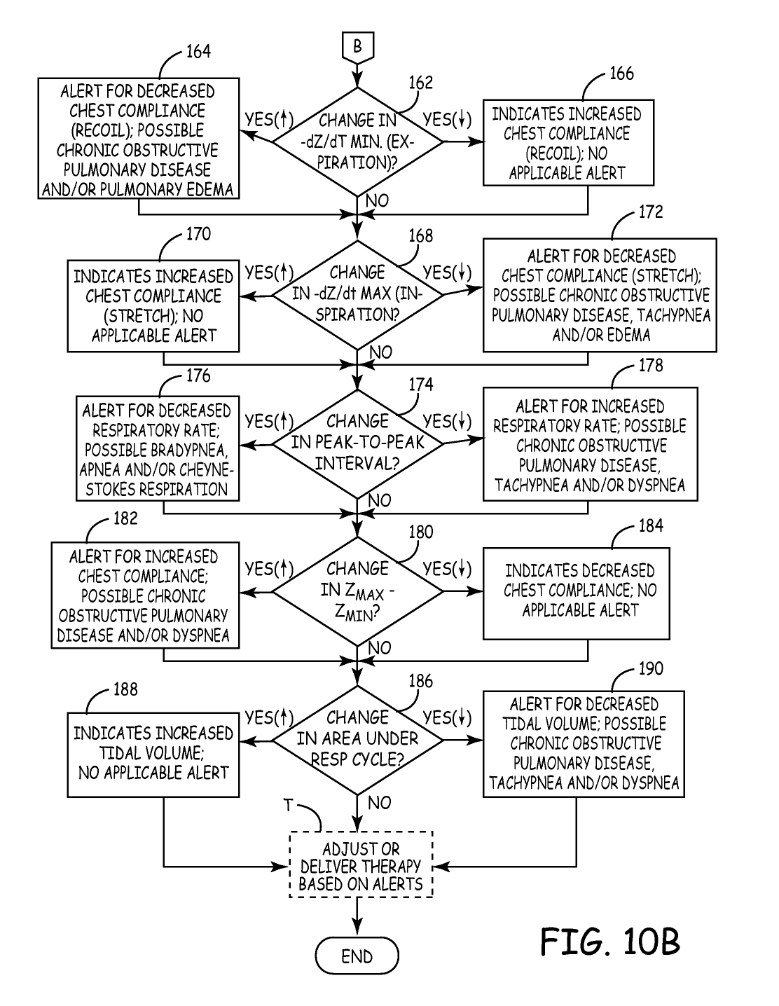

FIGS. 10A-10B together are a flow diagram illustrating a method of detecting changes in the morphology of a respiratory impedance waveform and providing alerts for changes in physiologic parameters based on the detected morphology changes. Upon starting the method (box 140), an electrode vector configuration is selected (box 142) to measure impedance in a tissue segment located in the vector field. For the method steps of FIGS. 10A-10B, an electrode vector configuration selected from the electrodes located as shown in FIG. 8 is selected. Impedance is then measured for a specified duration (box 144), and the impedance waveform is filtered to isolate the respiratory component of the impedance (box 146). This filtering step is achieved by filtering out the high frequency cardiac component of impedance with a low pass filter of some kind, or in some embodiments, this filtering step may be omitted (where the low frequency respiratory component may be analyzed effectively even with the high frequency cardiac component present). The impedance waveform is then analyzed to measure and store baseline impedance waveform information (box 148). This information may include (but is not limited to) a minimum peak impedance magnitude (Z_{MIN}), a maximum peak impedance magnitude (Z_{MAX}), a minimum to maximum impedance magnitude (Z_{MAX}-Z_{MIN}), a minimum negative slope (−dZ/dt) of the impedance waveform, a maximum positive slope (+dZ/dt) of the impedance waveform, a mean impedance, and a peak-to-peak time interval (which may involve a time interval between positive peaks (peaks), between negative peaks (nadirs), between a peak and a nadir, between a peak or a nadir and a characteristic of another monitored signal such as an ECG, between a point of maximum or minimum slope of the impedance waveform and a peak or a nadir, or others). Once this baseline impedance waveform information is determined and stored, changes in the impedance waveform with respect to the baseline may be detected and analyzed to provide alerts for various changes in physiologic parameters, as explained by the examples given below.

A change in the magnitude of impedance at end expiration may be detected as indicated at step 150, to monitor positive intrathoracic pressure during expiration. If the impedance magnitude has increased by an amount greater than a threshold, this indicates an increased positive end expiration intrathoracic pressure. In this case, an alert may accordingly be provided to indicate the increased positive end expiration intrathoracic pressure (box 152). This alert may provide an indication to a clinician of possible chronic obstructive pulmonary disease, for example. If the impedance magnitude has decreased by an amount greater than a threshold, this indicates decreased expiratory time. In this case, an alert may accordingly be provided to indicate the decreased expiratory time (box 154). This alert may provide an indication to a clinician of possible chronic obstructive pulmonary disease, tachypnea and/or dyspnea, for example.

A change in the magnitude of impedance at end inspiration may be detected as indicated at step 156, to monitor negative intrathoracic pressure during inspiration. If the impedance magnitude has increased by an amount greater than a threshold, this indicates decreased negative intrathoracic pressure. In this case, an alert may accordingly be provided to indicate the decreased negative intrathoracic pressure (box 158). This alert may provide an indication to a clinician of possible chronic obstructive pulmonary disease and/or dyspnea, for example. If the impedance magnitude has decreased by an amount greater than a threshold, this indicates increased negative intrathoracic pressure. In this case, an alert may accordingly be provided to indicate the increased negative intrathoracic pressure (box 160). This alert may provide an indication to a clinician of possible hypoxia, chronic obstructive pulmonary disease and/or dyspnea, for example.

A change in the minimum negative slope of the impedance waveform during expiration may be detected as indicated at step 162, to monitor thoracic cavity compliance (recoil). If the minimum negative slope has increased by an amount greater than a threshold, this indicates decreased chest compliance (recoil). In this case, an alert may accordingly be provided to indicate the decreased chest compliance (box 164). This alert may provide an indication to a clinician of possible chronic obstructive pulmonary disease and/or pulmonary edema, for example. If the minimum negative slope has decreased by an amount greater than a threshold, this indicates increased chest compliance (recoil). There is no applicable alert to be provided for this condition, as indicated by box 166.

A change in the maximum positive slope of the impedance waveform during inspiration may be detected as indicated at step 168, to monitor thoracic cavity compliance (stretch). If the maximum positive slope has increased by an amount greater than a threshold, this indicates increased chest compliance (stretch). There is no applicable alert to be provided for this condition, as indicated by box 170. If the maximum positive slope has decreased by an amount greater than a threshold, this indicates decreased chest compliance (stretch). In this case, an alert may accordingly be provided to indicate the decreased chest compliance (box 172). This alert may provide an indication to a clinician of possible chronic obstructive pulmonary disease and/or pulmonary edema, for example.

A change in the peak-to-peak interval (involving a time interval between positive peaks in the example given) of the impedance waveform may be detected as indicated at step 174, to monitor respiratory rate. If the peak-to-peak interval has increased by an amount greater than a threshold, this indicates decreased respiratory rate. In this case, an alert may accordingly be provided to indicate the decreased respiratory rate (box 176). This alert may provide an indication to a clinician of possible bradypnea, apnea and/or Cheyne-Stokes respiration, for example. If the peak-to-peak interval has decreased by an amount greater than a threshold, this indicates increased respiratory rate. In this case, an alert may accordingly be provided to indicate the increased respiratory rate (box 178). This alert may provide an indication to a clinician of possible chronic obstructive pulmonary disease, tachypnea and/or dyspnea, for example.

A change in the minimum-to-maximum value of impedance during a respiratory cycle may be detected as indicated at step 180, to monitor respiratory effort. If the minimum-to-maximum value of impedance has increased by an amount greater than a threshold, this indicates decreased chest compliance. In this case, an alert may accordingly be provided to indicate the decreased chest compliance (box 182). This alert may provide an indication to a clinician of possible chronic obstructive pulmonary disease and/or dyspnea, for example. If the minimum-to-maximum value of impedance has decreased by an amount greater than a threshold, there is no applicable alert to be provided, as indicated by box 184.

A change in the area under the impedance waveform for a respiratory cycle may be detected as indicated at step 186, to monitor tidal volume. If the area has increased by an amount greater than a threshold, this indicates increased tidal volume. There is no applicable alert to be provided for this condition, as indicated by box 188. If the area has decreased by an amount greater than a threshold, this indicates decreased tidal volume. In this case, an alert may accordingly be provided to indicate the decreased tidal volume (box 190). This alert may provide an indication to a clinician of possible chronic obstructive pulmonary disease, tachypnea and/or dyspnea, for example.

Although the description above indicates that a clinician reviews an alert indicating a change in a physiologic parameter to determine whether a clinical condition exists and a therapy may be needed, the system and method of the present invention may be employed to automatically trigger an alert for a clinical condition (or a number of possible clinical conditions) and to adjust or deliver an appropriate therapy, as desired for a particular patient environment. Box T illustrates the optional adjustment or delivery of therapy in response to generated alerts.

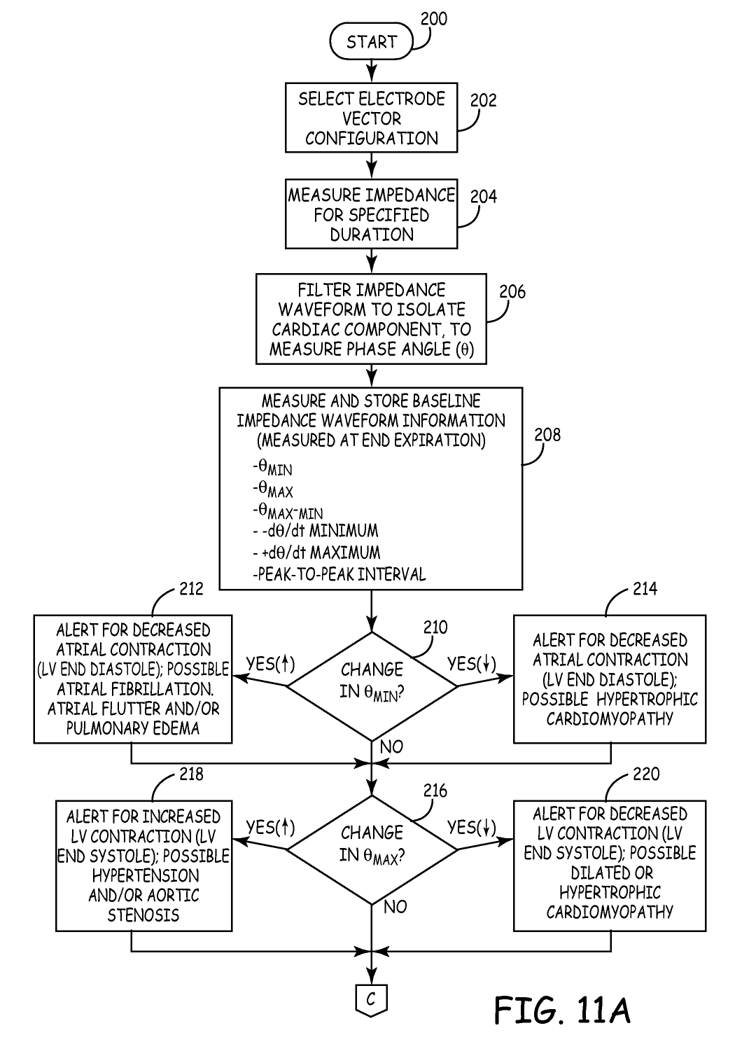

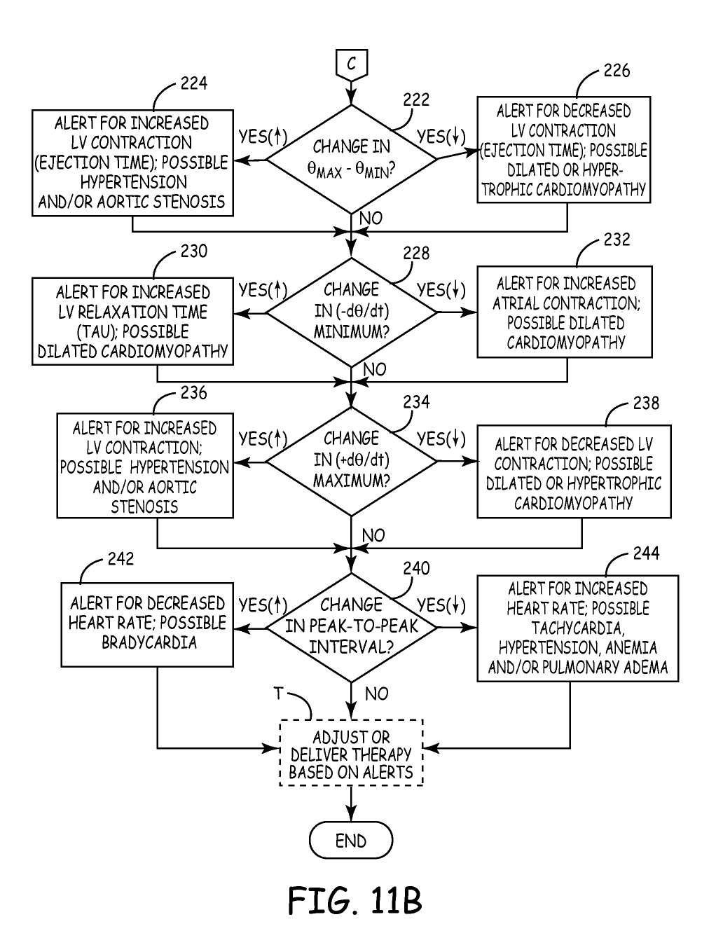

FIGS. 11A-11B together are a flow diagram illustrating a method of detecting changes in the morphology of the phase component of a cardiac impedance waveform and providing alerts for changes in physiologic parameters based on the detected morphology changes. Upon starting the method (box 200), an electrode vector configuration is selected (box 202) to measure impedance in a tissue segment located in the vector field. For the method steps of FIGS. 11A-11B, an electrode vector configuration selected from the electrodes located as shown in FIG. 8 is selected. Impedance is then measured for a specified duration (box 204), and the impedance waveform is filtered to isolate the cardiac component of the impedance (box 206), so that the cardiac impedance phase angle can be measured. This filtering step is achieved by filtering out the low frequency respiratory component of impedance with a high pass filter of some kind. The phase angle of the cardiac impedance waveform is then analyzed to measure and store baseline phase angle information (box 208). This information may include (but is not limited to) a minimum phase angle (θ_{MIN}), a maximum phase angle (θ_{MAX}), a minimum-to-maximum phase angle (θ_{MAX}-θ_{MIN}), a minimum negative slope (−dθ/dt) of the phase angle, a maximum positive slope (+dθ/dt) of the phase angle, and a peak-to-peak time interval (which may involve a time interval between positive peaks (peaks), between negative peaks (nadirs), between a peak and a nadir, between a peak or a nadir and a characteristic of another monitored signal such as an ECG, between a point of maximum or minimum slope of the phase angle of the impedance waveform and a peak or a nadir, or others). Once this baseline phase angle information is determined and stored, changes in the phase angle of the impedance waveform with respect to the baseline may be detected and analyzed to provide alerts for various changes in physiologic parameters, as explained by the examples given below.

A change in the minimum phase angle may be detected as indicated by step 210, to monitor atrial contraction at end diastole of the left ventricle. If the minimum phase angle increases by an amount greater than a threshold, this indicates decreased atrial contraction at end diastole of the left ventricle. In this case, an alert may accordingly be provided to indicate the decreased atrial contraction at end diastole (box 212). This alert may provide an indication to a clinician of possible atrial fibrillation, atrial flutter and/or pulmonary edema, for example. If the minimum phase angle decreases by an amount greater than a threshold, this indicates increased atrial contraction at end diastole of the left ventricle. In this case, an alert may accordingly be provided to indicate the increased atrial contraction at end diastole (box 214). This alert may provide an indication to a clinician of possible hypertrophic cardiomyopathy, for example.

A change in the maximum phase angle may be detected as indicated by step 216, to monitor left ventricle contraction at end systole. If the maximum phase angle increases by an amount greater than a threshold, this indicates increased left ventricle contraction at end systole. In this case, an alert may accordingly be provided to indicate the increased left ventricle contraction at end systole (box 218). This alert may provide an indication to a clinician of possible hypertension and/or aortic stenosis, for example. If the maximum phase angle decreases by an amount greater than a threshold, this indicates decreased left ventricle contraction at end systole. In this case, an alert may accordingly be provided to indicate the decreased left ventricle contraction at end systole (box 220). This alert may provide an indication to a clinician of possible dilated or hypertrophic cardiomyopathy, for example.

A change in the minimum-to-maximum phase angle may be detected as indicated by step 222, to monitor left ventricle contraction as reflected by ejection time. If the minimum-to-maximum phase angle increases by an amount greater than a threshold, this indicates increased left ventricle contraction. In this case, an alert may accordingly be provided to indicate the increased left ventricle contraction (box 224). This alert may provide an indication to a clinician of possible hypertension and/or aortic stenosis, for example. If the minimum-to-maximum phase angle decreases by an amount greater than a threshold, this indicates decreased left ventricle contraction. In this case, an alert may accordingly be provided to indicate the decreased left ventricle contraction (box 226). This alert may provide an indication to a clinician of possible dilated or hypertrophic cardiomyopathy, for example.

A change in the minimum negative slope of the phase angle may be detected as indicated by step 228, to monitor lusitropic function or relaxation of the left ventricle. If the minimum negative slope increases by an amount greater than a threshold, this indicates increased relaxation time (tau). In this case, an alert may accordingly be provided to indicate the increased relaxation time (box 230). This alert may provide an indication to a clinician of possible dilated cardiomyopathy, for example. If the minimum negative slope decreases by an amount greater than a threshold, this indicates increased atrial contraction. In this case, an alert may accordingly be provided to indicate the increased atrial contraction (box 232). This alert may provide an indication to a clinician of possible dilated cardiomyopathy, for example.

A change in the maximum positive slope of the phase angle may be detected as indicated by step 234, to monitor inotropic contractility of the left ventricle. If the maximum positive slope increases by an amount greater than a threshold, this indicates increased left ventricle contraction. In this case, an alert may accordingly be provided to indicate the increased left ventricle contraction (box 236). This alert may provide an indication to a clinician of possible hypertension and/or aortic stenosis, for example. If the maximum positive slope decreases by an amount greater than a threshold, this indicates decreased left ventricle contraction. In this case, an alert may accordingly be provided to indicate the decreased left ventricle contraction (box 238). This alert may provide an indication to a clinician of possible dilated or hypertrophic cardiomyopathy, for example.

A change in the peak-to-peak interval of the phase angle (involving a time interval between positive peaks in the example given) may be detected as indicated by step 240, to monitor heart rate. If the peak-to-peak interval increases by an amount greater than a threshold, this indicates decreased heart rate. In this case, an alert may accordingly be provided to indicate the decreased heart rate (box 242). This alert may provide an indication to a clinician of possible bradycardia, for example. If the peak-to-peak interval decreases by an amount greater than a threshold, this indicates increased heart rate. In this case, an alert may accordingly be provided to indicate the increased heart rate (box 244). This alert may provide an indication to a clinician of possible tachycardia, hypertension, anemia and/or pulmonary edema, for example.

Although the description above indicates that a clinician reviews an alert indicating a change in a physiologic parameter to determine whether a clinical condition exists and a therapy may be needed, the system and method of the present invention may be employed to automatically trigger an alert for a clinical condition (or a number of possible clinical conditions) and to adjust or deliver an appropriate therapy, as desired for a particular patient environment. Box T illustrates the optional adjustment or delivery of therapy in response to generated alerts.

For each of the physiologic parameters described as being monitored in FIGS. 9A-9B, 10A-10B and 11A-11B, there are a number of changes in the impedance waveform morphology that may be used to trigger an alert for review by a clinician. A change from a stored baseline characteristic that exceeds a threshold has been described. In addition, an absolute value of an impedance waveform characteristic that falls outside of a prescribed range, or certain long-term trends in an impedance waveform characteristic, for example, may also trigger an alert for review by a clinician (or for automatic adjustment or delivery of therapy in some embodiments).

The discussion above indicates that in exemplary embodiments, impedance is measured by measuring voltage and dividing the value of the voltage by the value of the injection current to derive the value of impedance. It should be understood that in other embodiments, it may be possible to simply measure voltage, and to monitor the measured voltage for changes in order to detect changes in physiologic parameters, by making an assumption that the voltage changes will reflect the impedance changes in the tissue being monitored. Thus, references to measuring impedance herein encompass a variety of methods to measure electrical parameters related to impedance, including simply measuring voltage in some embodiments.

The examples of physiologic parameters and clinical conditions are provided as examples of parameters that can be monitored using selected electrodes in the electrode vector configuration shown in FIG. 8. Other physiologic parameters, related to other clinical conditions, may be monitored and used to provide alerts for other electrode vector configurations. Examples of a number of other useful electrode vector configurations are given in U.S. application Ser. No. ______ filed on even date herewith, for “Multi-Frequency Impedance Monitoring System” by T. Zielinski, D. Hettrick and S. Sarkar.

Although the present invention has been described with reference to preferred embodiments, workers skilled in the art will recognize that changes may be made in form and detail without departing from the spirit and scope of the invention.

1. A method of monitoring physiologic parameters in a patient, the method comprising:
measuring impedance of a tissue segment located in a selected electrode vector field;
storing baseline impedance information based on the measured impedance;
detecting changes in impedance characteristics from the baseline impedance information; and
providing alerts indicating changes in the physiologic parameters based on the detected changes in impedance characteristics.

2. A method according to claim 1, wherein measuring the impedance of the tissue segment in the selected electrode vector field comprises measuring a real component and a reactive component of the impedance.

3. A method according to claim 1, wherein detecting changes in impedance characteristics from the baseline impedance information includes detecting changes in morphology of an impedance waveform.

4. A method according to claim 1, further comprising filtering the measured impedance of the tissue segment to isolate a cardiac component of the impedance.

5. A method according to claim 4, wherein the baseline impedance information includes at least one of a minimum impedance, a maximum impedance, a minimum-to-maximum impedance difference, a maximum positive rate of change of impedance, a minimum negative rate of change of impedance, a mean impedance, and a time interval between impedance peaks.

6. A method according to claim 5, wherein the physiologic parameters include at least one of left ventricle end diastolic volume at end expiration, left ventricle end systolic volume at end expiration, stroke volume, left ventricle lusitropic function/relaxation, left ventricle inotropic contractility, fluid status in the electrode vector field, and heart rate.

7. A method according to claim 1, further comprising filtering the measured impedance of the tissue segment to isolate a respiratory component of the impedance.

8. A method according to claim 7, wherein the baseline impedance information includes at least one of an impedance magnitude at end expiration, an impedance magnitude at end inspiration, a minimum negative rate of change of impedance during expiration, a maximum positive rate of change of impedance during inspiration, a time interval between impedance peaks, a minimum-to-maximum impedance during a respiratory cycle, and an area under an impedance waveform during a respiratory cycle.

9. A method according to claim 8, wherein the physiologic parameters include at least one of positive intrathoracic pressure during expiration, negative intrathoracic pressure during inspiration, thoracic cavity compliance (recoil), thoracic cavity compliance (stretch), respiratory rate, respiratory effort, and tidal volume.

10. A method according to claim 1, further comprising determining a phase angle of the measured impedance of the tissue segment.

11. A method according to claim 10, wherein the baseline impedance information includes at least one of a minimum phase angle, a maximum phase angle, a minimum-to-maximum phase angle difference, a minimum negative rate of change of phase angle, a maximum positive rate of change of phase angle, and a time interval between phase angle peaks.

12. A method according to claim 11, wherein the physiologic parameters include at least one of atrial contraction at left ventricle end diastole, left ventricle contraction at end systole, left ventricle contraction as reflected by ejection time, left ventricle lusitropic function/relaxation, inotropic contractility of the left ventricle, and heart rate.

13. A method according to claim 1, wherein measuring the impedance of the tissue segment located in the selected electrode vector field comprises:
positioning a plurality of electrodes in the patient cutaneously, subcutaneously, intravascularly, intracardially, or any combination of these;
injecting a current between selected electrodes of the plurality of electrodes; and
measuring a voltage between selected electrodes of the plurality of electrodes to determine an impedance of a tissue segment located in the electrode vector field therebetween as a function of the injected current and the measured voltage.

14. A method according to claim 1, further comprising adjusting or delivering therapy based on alerts provided to indicate changes in the physiologic parameters.

15. A method of monitoring a physiologic parameter in a patient, the method comprising:
positioning a plurality of electrodes in the patient cutaneously, subcutaneously, intravascularly, intracardially, or any combination of these;
selecting an electrode vector from the plurality of electrodes to create an electrode vector field that includes a tissue segment such that a change in impedance in the electrode vector field reflects a change in the physiologic parameter being monitored;
measuring impedance of the tissue segment located in the selected electrode vector field;
storing baseline impedance information based on the measured impedance;
detecting changes in impedance characteristics from the baseline impedance information; and
providing alerts indicating changes in the physiologic parameters based on the detected changes in impedance characteristics.

16. A method according to claim 15, wherein measuring impedance of the tissue segment in the selected electrode vector field comprises measuring a real component and a reactive component of the impedance.

17. A method according to claim 15, wherein measuring the impedance of the tissue segment located in the selected electrode vector field comprises:
injecting a current between the selected electrodes of the plurality of electrodes; and
measuring a voltage between the selected electrodes to determine the impedance of the tissue segment located in the electrode vector field therebetween as a function of the injected current and the measured voltage.

18. A method according to claim 15, wherein detecting changes in impedance characteristics from the baseline impedance information includes detecting changes in morphology of an impedance waveform.

19. A method according to claim 15, further comprising filtering the measured impedance of the tissue segment to isolate a cardiac component of the impedance, wherein the baseline impedance information includes at least one of:
a minimum impedance, a maximum impedance, a minimum-to-maximum impedance difference, a maximum positive rate of change of impedance, a minimum negative rate of change of impedance, a mean impedance, and a time interval between impedance peaks, and
the physiologic parameter comprises at least one of:
a left ventricle end diastolic volume at end expiration, a left ventricle end systolic volume at end expiration, a stroke volume, a left ventricle lusitropic function/relaxation, a left ventricle inotropic contractility, a fluid status in the electrode vector field, and a heart rate.

20. A method according to claim 15, further comprising filtering the measured impedance of the tissue segment to isolate a respiratory component of the impedance, wherein the baseline impedance information includes at least one of:
an impedance magnitude at end expiration, an impedance magnitude at end inspiration, a minimum negative rate of change of impedance during expiration, a maximum positive rate of change of impedance during inspiration, a time interval between impedance peaks, a minimum-to-maximum impedance during a respiratory cycle, and an area under an impedance waveform during a respiratory cycle, and
the physiologic parameter comprises at least one of:
a positive intrathoracic pressure during expiration, a negative intrathoracic pressure during inspiration, a thoracic cavity compliance (recoil), a thoracic cavity compliance (stretch), a respiratory rate, a respiratory effort, and a tidal volume.

21. A method according to claim 15, further comprising determining a phase angle of the measured impedance of the tissue segment, wherein the baseline impedance information includes at least one of a minimum phase angle, a maximum phase angle, a minimum-to-maximum phase angle difference, a minimum negative rate of change of phase angle, a maximum positive rate of change of phase angle, and a time interval between phase angle peaks, and the physiologic parameter comprises at least one of atrial contraction at left ventricle end diastole, left ventricle contraction at end systole, left ventricle contraction as reflected by ejection time, left ventricle lusitropic function/relaxation, inotropic contractility of the left ventricle, and heart rate.

22. A method according to claim 15, further comprising adjusting or delivering therapy based on alerts provided to indicate changes in the physiologic parameter.

23. An apparatus for monitoring physiologic parameters in a patient, comprising:
means for measuring impedance of a tissue segment located in a selected electrode vector field;
means for storing baseline impedance information based on the measured impedance;
means for detecting changes in impedance characteristics from the baseline impedance information; and
providing alerts indicating changes in the physiologic parameters based on the detected changes in impedance characteristics.

24. An apparatus according to claim 23, wherein the means for measuring the impedance of the tissue segment in the selected electrode vector field comprises means for measuring a real component and a reactive component of the impedance.

25. An apparatus according to claim 23, wherein the means for detecting changes in impedance characteristics from the baseline impedance information includes means for detecting changes in morphology of an impedance waveform.

26. An apparatus according to claim 23, further comprising means for filtering the measured impedance of the tissue segment to isolate a cardiac component of the impedance.

27. An apparatus according to claim 26, wherein the baseline impedance information includes at least one of:
a minimum impedance, a maximum impedance, a minimum-to-maximum impedance difference, a maximum positive rate of change of impedance, a minimum negative rate of change of impedance, a mean impedance, and a time interval between impedance peaks.
